(19) United States
(12) Patent Application Publication
(10) Pub. No.: US 2022/0370645 A1
(43) Pub. Date: Nov. 24, 2022

(54) METHOD, DEVICE AND MARKER SUBSTANCE KIT FOR MULTI-PARAMETRIC X-RAY FLUORESCENCE IMAGING

(71) Applicant: Universitaet Hamburg, Hamburg (DE)

(72) Inventor: Florian GRUENER, Hamburg (DE)

(21) Appl. No.: 17/771,460

(22) PCT Filed: Oct. 23, 2020
(86) PCT No.: PCT/EP2020/079909

§ 371 Date: Apr. 23, 2022

(30) Foreign Application Priority Data
Oct. 25, 2019 (DE) 10 2019 128 842.8

(51) Int. Cl. A61K 49/04; G01N 23/22

(52) U.S. Cl. CPC A61K 49/0423; G01N 23/22; G01N 2223/076

(57) ABSTRACT

A method for multi-parametric x-ray fluorescence imaging with maximized detection sensitivity and minimized radiation dose for a biological/living sample (10) containing a first marker substance comprises the steps of irradiation of the sample (10) with x-ray radiation (1), with x-ray fluorescence (2) of the first marker substance being excited, spatially resolved detection of the x-ray fluorescence (2) of the first marker substance, and determination of a distribution of the first marker substance in the sample (10) from the x-ray fluorescence (2) of the first marker substance, wherein the sample (10) contains at least one further marker substance which is excited to x-ray fluorescence (2) by the x-ray radiation (1), wherein fluorescence lines (3) of the first and the at least one further marker substances are different, at least one of the first and the at least one further marker substances is coupled with active ingredient molecules and/or ligand molecules provided for a specific interaction with the sample (10) or contained in cells, in order to be able to trace these, the detection comprises a spectrally resolved detection of the x-ray fluorescence (2) of the first and the at least one further marker substances, and additionally at least one distribution of the at least one further marker substance in the sample (10) is determined from the detected x-ray fluorescence (2) of the first and the at least one further marker substances. An imaging device (100) for multi-parametric x-ray fluorescence imaging and an optimized selection method for a marker substance kit for introducing marker substances into a sample (10) are also described.

The invention relates to a method for multi-parametric X-ray fluorescence imaging on a sample, in which marker substance distributions in the sample are detected. The invention furthermore relates to an imaging apparatus which is configured for X-ray fluorescence imaging on a sample to be investigated, and to a marker substance kit which is configured for introduction into a sample for X-ray fluorescence imaging on the sample. The invention can be used in X-ray fluorescence imaging for samples, in particular biological samples or non-biological samples.

In the present description, reference is made to the following prior art, which represents the technical background of the invention:
[1] DE 10 2017 003 517;
[2] US 2012/0307962 A1;
[3] US 2016/0252471 A1;
[4] T. Pellegrino et al. in “Nano Lett.” Vol. 4, No. 4, 2004, p. 703-707;
[5] H. D. Fiedler et al. in “Anal Chem.” 2013, 85(21):10142-8; and
[6] R. Zhang et al. in “Am. J. Nucl. Med. Mol. Imaging” 2018, 8(3):169-188;
[7] F. Grüner et al. in “Sci. Rep.” Vol. 8, 2018, p. 16561;
[8] K. Khrennikov et al. in “Phys. Rev. Lett.” Vol. 114, p. 195003 (2015);
[9] M. Hossain et al. in “Applied Physics Letters” Vol. 97, 2010, p. 263704-1-263704-3;
[10] D. P. Cormode et al. in “Contrast Media & Molecular Imaging” 2014, No. 9, p. 37-52; and
[11] Y. Li et al. in “Contrast Media & Molecular Imaging” Vol. 2018, 2018, Article ID 8174820, p. 1-7 (with Supplementary material).

It is of interest, in pharmacology, to measure the local and/or temporal distribution of physiologically acting substances (also referred to here as active substances or active substance molecules or pharmacologically active substances), in particular biomarkers, antibodies, antibody fragments, biological cells, and/or medicaments (medicament molecules), in the body of a patient or a test animal (measurement of the pharmacokinetics). The measurement of the pharmacokinetics aims in particular to acquire the local concentration of applied active substances in the body in a time-dependent manner, because the effectiveness of the active substances is directly dependent on the concentration (and time period) in which they are bound at the therapeutic site of action, e.g. by coupling to receptors on cell surfaces. Conventional methods of pharmacokinetics are of limited conclusiveness. If, for example, following administration of a medicament, blood samples are taken in order to measure the concentration of the medicament in the blood, no local distribution of the medicament in the body is acquired, and in particular no information is obtained with regard to whether, when, and at what concentration the medicament has reached the desired site of action. If a plurality of active substances is applied, which is referred to here in general as multi-medication, these can be acquired separately only if laborious method steps are applied.

A further important application would be the measurement of the distribution of different immune cell types, for example in order to record the effectiveness of medicaments for the treatment of Crohn's disease and/or ulcerative colitis, as well as further immune system-mediated inflammatory diseases. Since, in this case, a plurality of different immune cell types is usually relevant, the hitherto unresolved challenge arises of measuring the dynamics of the different immune cell types in the body separately from one another, but simultaneously and at the same location.

Positron emission tomography (PET) is a generally known method by means of which it is possible to record the distribution for example of a medicament in the body. PET is based on a radioactive tracer molecule being bound to the medicament molecule. The tracer molecule emits a positron, in the body under investigation, which annihilates with an electron, as a result of which two X-ray photons having a characteristic energy of 511 keV are generated. These two X-ray photons are detected, it being possible for the emission location, and thus the location of the medicament, to be determined by a plurality of coincidence measurements. However, PET has a series of disadvantages, which result from the radiation exposure of the body, the complex instrument technique, and limited conclusiveness in the case of multi-medication.

In the case of multi-medication, PET delivers limited information, since only one single medicament distribution can be recorded by means of one measurement (no simultaneous pharmacokinetics of a plurality of active substances). Even if a plurality of medicaments is applied simultaneously, these cannot be measured separately. Since, in the case of all annihilation events, only photons having an energy of 511 keV can be generated, even different tracer molecules, in conjunction with different medicament molecules in each case, could not be distinguished, and thus no medicament-specific emission locations could be detected. Although PET offers the possibility of sequential measurement, in that initially a first medicament is injected and detected, and subsequently a second medicament is injected and detected, this is impractical because, in the case of PET, the diagnostic time window is very restricted, on account of the rapid decay of the tracer molecules. Thus, application of PET in the case of multi-medication is ruled out in practice.

A further generally known functional imaging method is that of single-photon emission computerized tomography (SPECT) which, however, likewise cannot be used for detecting active agent distributions in the case of multi-medication. Although, in the case of SPECT, different tracer molecules could be used, which would be distinguishable on account of different emission energies, the tracer molecules can be bound to biomolecules only in a very complex manner (by means of radiochemistry). Furthermore, available tracer molecules for SPECT have very different emission energies and half-life periods, and therefore can be detected together only with difficulty—the efficiency of detectors is greatly dependent on the energy of the incoming photons, and the number of photons emitted in each case reduces to a different extend, depending on the half-life period. Finally, available tracer molecules offer only restricted diagnostic time windows, which would usually be too short for investigating temporal active substance distributions. A SPECT-based solution would be significantly more complex, because the binding of SPECT tracer molecules to the medicament molecules is difficult, in particular in the case of different tracer molecules which are intended to be used simultaneously. A determination of the activity directly before the injection, which is indispensable for the quantitative evaluation, would also be difficult in this case.

Even a combination of PET with SPECT could not overcome the limitations of PET, because, although in the case of simultaneous use of two different tracer molecules both methods could also track two different medicaments, this would be possible only over their diagnostic time windows which are significantly restricted by the respective half-life periods. Furthermore, hitherto no combination devices are known which allow for both PET and SPECT simultaneously, because the two methods operate differently.

X-ray fluorescence imaging (XFI) is a further method for detecting the distribution of a medicament in the body (see e.g. [1] to [3], [9] and [11]). The diagnostic X-ray fluorescence imaging is based on applying a marker substance, comprising for example a plurality of nanoparticles, in a body to be investigated, and performing a detection, in a spatially resolved manner, by means of induced fluorescence in the X-ray wavelength range. If ligands are bound to the nanoparticles (functionalized nanoparticles) and the ligands are connected to or themselves comprise an active substance, such as antibodies or antibody fragments, biological cell, biomarkers, or medicaments, information relating to the distribution of the active substance in the body is obtained by means of the spatially resolved measurement of the X-ray fluorescence. In XFI studies, gold nanoparticles are usually used, because these are easy to synthesize, and their functionalization is based on well-studied coupling chemistry. Further nanoparticles are described in [4], [5], [9], [10] (in connection with computerized tomography) and [11]. It is known from [6] to detect, by means of XFI, a plurality of different toxic metals, already present in an organism prior to the measurement, on account of their respective X-ray fluorescence in the organism.

However, it is known from studies using XFI or invasive methods that even non-functionalized nanoparticles collect at different concentrations in different organs of biological organisms. Furthermore, nanoparticles have a strongly asymmetrical mass ratio compared with typical medicament molecules, and therefore the transport of the medicament in the body could be dominated by that of the nanoparticles. These properties of nanoparticles restrict the conclusiveness of the conventional XFI method: If the concentration of gold nanoparticles in a particular organ is measured using XFI, the gold nanoparticles being functionalized with a medicament, it is not possible to conclude, on the basis of the measured concentration value alone, that this was determined by the medicament. It could be possible for the gold nanoparticles to have also reached the same concentration without the bound medicament.

It is therefore proposed, in [7], for comparison purposes, to measure nanoparticles with ligands, in a first organism, and nanoparticles without ligands in a second, separate organism (in this case mice). However, this method leads to a high requirement of test animals, and it ignores the individual differences between individual organisms. Furthermore, it is not practical in medicine for patients. It would therefore be of interest to perform the comparison measurement simultaneously in one single organism. This comparison measurement would furthermore allow for the detection of the unspecific, physiological background in the target region. If for example blood vessels, and, therein, unbound nanoparticles, are located here, this “background image” can be subtracted from an XFI image. The difference image then shows only the specifically bonded nanoparticles. This comparison measurement, which cannot be performed using conventional techniques, would furthermore allow for the detection of the unspecific, physiological background in the target region. If for example blood vessels, and, therein, unbound nanoparticles, are located here, this “background image” can be subtracted from an XFI image. The difference image then shows only the specifically bonded nanoparticles.

A further disadvantage of the conventional XFI is that, in the case of multi-medication, it is not possible to track a plurality of different medicaments or biomarkers simultaneously. This also applies for the simultaneous imaging of the dynamics of different immune cell types. However, measurements of this kind would be important in a series of medical studies, such as for visualization of inflammation processes in the body, or for tumor diagnosis using a plurality of different antibodies. For example, in the case of inflammation processes different immune cell types are intended to be recorded, which arrive at the inflammation site at different times and thus influence the progression of the inflammation. It is thus of interest, in the case of Crohn's disease, to track four different immune cell types simultaneously. However, different cell types cannot be distinguished by means of conventional XFI. The measurement of the pharmacokinetics in the case of multi-medication would also be of interest, in order to investigate the interaction of medicaments or compare the effect of medicaments. It is generally known that new medicaments often fail only after being brought onto the market, because patients have to take several medicaments at the same time, which block binding sites, for one another, in the body. Hitherto, this aim is not achievable, because conventional techniques do not make it possible for all the immune cell types, which are to be measured simultaneously, to be determined at an identical sensitivity.

A further important question in the case of conventional XFI is the problem that it is know that the kinetics of the nanoparticles depends on their size. A practical and effective method for tracking the kinetics of a plurality of nanoparticle types of different sizes, together and at the same sensitivity, and for directly comparing these with one another, is not currently available.

For the purpose of measuring the pharmacokinetics in the case of multi-medication, a sequential measurement could be performed also in the case of XFI, but this would require unacceptably long measuring times. Different medicaments could only be investigated in succession after an investigated medicament has, in each case, been broken down and egested. It is thus not possible to measure multi-parametric pharmacokinetics, under practical routine conditions, by means of conventional XFI.

In [9], a multiplex biomarker detection by means of X-ray fluorescence, of different nanoparticle types, is described. The authors of [9] have identified that the different nanoparticle types can be distinguished on the basis of their different spectral fluorescence properties, and that a spatially resolved measurement of different nanoparticle types at different locations is possible. The technique according to [9] was studied on the basis of a model system in which the nanoparticles are irradiated with high excitation intensities of the exciting X-ray radiation, on a thin aluminum substrate. The high excitation intensities make it possible to generate sufficiently high fluorescence intensities of the X-ray fluorescence, and to thus be able to detect this sensitively. The use of the aluminum substrate makes it possible to largely avoid interfering scattered radiation (background radiation) on account of multiple scatterings.

However, the technique according to [9] is not applicable in biomedical imaging, because, for reasons of radiation protection, significantly lower intensities of the exciting X-ray radiation are required in this technique, and significantly stronger scattered radiation is generated in the biological tissue, because the objects (such as a mouse) present significantly more tissue, in which more (multiple) scattering arises. In order to overcome this problem it is proposed, in [9], to use monochromatic X-ray radiation for the excitation, which, however, is found in practice to be insufficient for achieving sufficient sensitivity of the X-ray fluorescence detection, in particular for objects which are significantly larger than the substrate used in [9], because, on account of their larger volume, the scattered radiation results in such a large background in the detected spectrum that weak XFI signals can no longer be detected, at a minimum radiation dose. A targeted selection of the marker kit is not described in [2] either.

In [2], a method for computerized tomography XFI is described, in which the XFI can in principle take place using a composition of a plurality of different nanoparticle types. In this case, however, the spectrally different emissions of the different nanoparticle types, in the evaluation of the detected X-ray fluorescence, are not used for imaging on living organisms.

A library of nanoparticles for X-ray fluorescence tomography is described in [11]. The authors of [11] have shown in the supplementary material of [11], that, in the case of nanoparticles consisting of different elements, in each case intensities of the scattered radiation occur which vary over several orders of magnitude, such that the different nanoparticles cannot be detected at the same levels of sensitivity. Overcoming this problem in XFI on biological samples by means of adapting the concentration of the nanoparticles, in that the concentration of nanoparticles having a high background is increased for example by a factor of 1000, is subject to strict physiological boundaries. It is therefore proposed in [11], as a selection criterion for the combination of nanoparticles, that the K edges of the emitting elements of the nanoparticles should be as close as possible to the (average) energy of the X-ray source. However, it is precisely this that does not allow for minimization of the background. Therefore, in [11], the background in the signal range of the studied marker elements is both very different and also not minimized, overall. Highly sensitive XFI is therefore not possible.

An objective of the invention is to provide an improved method for X-ray fluorescence imaging on a sample, by means of which disadvantages of conventional techniques are avoided. A further objective of the invention is to provide an improved imaging apparatus for X-ray fluorescence imaging for investigating a sample, wherein disadvantages of conventional techniques are avoided by means of the imaging apparatus. A further objective of the invention is to provide an improved marker substance kit which is adapted for an introduction into a sample for X-ray fluorescence imaging on the sample. The invention is intended in particular to provide highly sensitive XFI having increased conclusiveness, to allow for the measurement of a spatial and/or temporal distribution of one or multiple active substances in a body of an investigated organism, and/or to provide new applications of XFI, in particular under practical application conditions. In this case, for example in vivo imaging comprising detection of one or multiple active substances, in particular biomarkers, antibodies, antibody fragments, biological cells, such as immune cells, and/or medicaments, is of particular interest. The XFI is intended in particular to allow for study in the case of multi-medication with reduced method complexity, and/or to prevent restrictions to short diagnostic time windows.

These objectives are achieved in each case by a method for X-ray fluorescence imaging, an X-ray fluorescence imaging apparatus, and a marker substance kit, which have the features of the independent claims. Preferred embodiments and applications of the invention can be found in the dependent claims.

According to a first general aspect of the invention, the above objective is achieved by a method for multi-parametric X-ray fluorescence imaging of a sample, wherein the following steps are provided. The sample to be investigated is irradiated with X-ray radiation, preferably with monochromatic or at least narrow-band X-ray radiation, wherein X-ray fluorescence of a first marker substance is excited. The sample is generally a shape-retaining object, preferably a body (or a part of the body) of a biological organism, particularly preferably a body (or a part of the body) of a human or animal subject. Alternatively, other non-natural objects, such as synthetic biological objects or artificial organs, such as implants or skin models, or technical objects, can also be investigated. A spatially resolved detection of the X-ray fluorescence of the first marker substance takes place, in particular by scanning the sample. A distribution of the first marker substance in the sample can be determined from the detected X-ray fluorescence of the first marker substance.

According to the invention, the sample contains, in addition to the first marker substance, at least one further marker substance, which is excited to X-ray fluorescence by the X-ray radiation. The excitation of the X-ray fluorescence preferably takes place simultaneously, if the at least one further marker substance is located in the same irradiated region (beam volume at a position of the irradiation, scan position) like the first marker substance. The marker substances are molecular or particulate substances which in each case contain at least one X-ray fluorescence element as a pure element or in a chemical compound. The marker substances are substances that are foreign to the body and have been purposely supplied to the sample prior to the XFI method, and leave the sample again, after a substance-specific dwell time, after the XFI method, e.g. are separated out by transport processes. Active substance molecules and/or ligand molecules which exhibit a specific biological and/or chemical interaction with the sample, and are transported in particular portions of the sample, by means of transport processes for example, such as metabolism or blood transport, and/or couple therein, are coupled to at least one of the first and at least one further marker substance. If, according to a preferred variant, the active substances comprise biological cells, e.g. for the immune cell therapy, and in particular in an application in which the cells are intended to be monitored by means of the method according to the invention, the coupled marker substances are preferably contained in the cells or are bound to the surface thereof. The first and each further marker substance are characterized by different X-ray fluorescence in each case. In a measured spectrum, the fluorescence lines of the X-ray fluorescence elements of the first and each further marker substance are different in each case, and they in particular have maxima at different energies and/or different spectral widths. According to the invention, the detection comprises a spectrally and spatially resolved acquisition of the X-ray fluorescence (X-ray fluorescence emissions) of the first and each further marker substance, preferably in a single measuring procedure, e.g. by scanning using an X-ray beam. In addition to the distribution of the first marker substance, at least one distribution of the at least one further marker substance in the sample is determined from the detected X-ray fluorescence of the first and each further marker substance.

As a result, this multi-parametric method delivers multiple specific distributions of the first and each further marker substance in the sample. The determined distributions typically do not constitute diagnostic information per se. A medical diagnosis on the sample can separately follow the method according to the invention, the determined distributions being used.

The first and the at least one further marker substance preferably exhibit fluorescence probabilities, attenuations of the X-ray fluorescence in the sample, and background noise levels in the sample which are the equal or are similar to such an extent that the detection of the X-ray fluorescence at the same concentration of the marker substances results in comparable statistical significance levels. Furthermore, the irradiation photon energy of the exciting X-ray radiation is preferably above the absorption edges of all the marker substances. The term “concentration” refers to the mass of a marker substance per (beam) surface in the sample, i.e. to the “areal density”. The “fluorescence probability” refers to the effective cross section of the individual atoms of the marker substance. The number of fluorescence photons generated by a marker substance which delivers the detectable signal following transmission of the fluorescence through the sample, is determined by the product of the areal density and the effective cross section for fluorescence of the relevant marker substance. If the effective cross sections of two marker substances, and also the transmissions, are equal or similar, and the areal densities are equal or similar, this advantageously delivers equal or similar detectable signals of the marker substances.

The marker substances preferably comprise nanoparticles. The nanoparticles particularly preferably comprise outer surfaces which are indistinguishable for the sample, are preferably identical, can differ merely by targeted functionalization, and the inside of which consists of different elements, where each has its characteristic “fluorescence fingerprint”. These nanoparticles may have been introduced into the sample from the outside, as in the case of conventional nanoparticle-based XFI.

In particular, the targeted selection of the X-ray fluorescence elements of the marker substances is advantageous for the multi-parametric XFI. This selection preferably fulfils the following criteria:

(a) the irradiation photon energy is above all the absorption edges,

(b) the X-ray fluorescence elements exhibit a similar fluorescence probability,

(c) the attenuation of the respective fluorescence lines of the X-ray fluorescence elements should also be similar,

(d) the fluorescence lines of the X-ray fluorescence elements should have similar background noise levels on account of scattering (single and/or multiple scattering) in the sample, such that, together with (b) and (c), the respective statistical significances of the detected fluorescence lines of the elements have similar levels, if the areal densities (concentrations) of the elements in the X-ray volume are the same.

For example, the ratio of the number of recorded fluorescence photons to the root of the number of background photons, or a variable which is quantitatively representative of this ratio, can be employed as a measure for the level of the statistical significance, i.e. in particular for the sensitivity of the X-ray fluorescence detection. The root of the number of background photons is in turn a measure for the statistical background noise. Both the number of the fluorescence photons, and that of the background, can be determined by mathematical fit functions of the measured X-ray spectrum in the region of the fluorescence lines, using available numerical methods.

The criteria (b) to (d) are a particularly important finding of the inventors, and are particularly advantageous for implementing embodiments of the invention—if for example two X-ray fluorescence elements are selected which are so far apart in the periodic system that their fluorescence probabilities (i.e. effective cross sections), attenuations in the sample, and background noise levels are excessively different, the two would be detected at significantly different sensitivities, even if the areal density of both elements in the X-ray volume were identical. This could result in only one of the two marker substance types being effectively detectable, with the result that multi-parametric XFI could not be achieved.

The effective cross sections of the atoms of the marker substances are known from standard measurements and published tables, and the attenuations of the X-ray fluorescence in the sample can be determined by reference measurements and/or simulations. The background is formed both by scattering of the incoming photons in the sample, and by detector effects. The background behavior of X-ray fluorescence elements can be determined by numerical simulations or measurements (see e.g. [7]), in order to thereby make an optimal selection of the X-ray fluorescence elements according to the criteria (b) to (d).

The introduction of the marker substances into the sample is not considered part of the invention, insofar as the introduction requires an invasive intervention into biological material, such as an injection.

According to a preferred embodiment of the invention, the statistical significance levels of the marker substances are maximized in that the irradiation photon energy of the X-ray radiation is selected so as to be of a spacing above the highest absorption edge of the marker substances in the sample, such that the background noise levels of the marker substances are minimal and equal or approximately equal, and at the same time the effective cross sections and the transmissions of the X-ray fluorescence through the sample are maximum and equal or approximately equal. The mentioned variables are preferably approximately equal, if differences in the background noise levels or the effective cross sections and the transmissions have a negligibly small influence on the detection of the X-ray fluorescence.

The selection of the irradiation photon energy of the X-ray radiation, and the selection of the marker substances, is preferably performed by means of an optimization, wherein the irradiation photon energy is selected so as to be as large as possible, compared with the highest absorption edge of the marker substances in the sample, in order that incoming photons have to be scattered as much as possible and suffer corresponding energy losses until they fall into the energy range of the X-ray fluorescence lines (minimization of the background noise by necessary multiple scattering, which becomes less likely the more scatterings are required for the total energy lost, from the irradiation energy down to the fluorescence energy range). At the same time, the irradiation photon energy should not be selected so as to be so high that the effective cross sections are reduced too greatly.

The invention provides, for the first time, multi-parametric X-ray fluorescence imaging on a sample which is suitable for practical application. In [9] although, as described above, two different nanoparticles are already used, these are used only in vitro on a very thin substrate. In contrast, the present invention allows, by means of the selection of the marker substances, which is not described in [9], reliable XFI on real objects, such as objects of at least the size of a mouse. The selection, according to the invention, of the marker substances is not described in [2] or [11] either, and therefore for example the XFI marker substances mentioned in [11] had very different imaging sensitivities, and effective multi-parametric XFI is excluded.

Unlike in the case of the above-mentioned methods, described in [7], the invention allows for comparison measurements on a single sample, because the simultaneous comparison measurement has the same imaging sensitivity as the actual measurement, such that clear results are obtained.

According to a second general aspect of the invention, the above objective is achieved by an imaging apparatus which is configured for multi-parametric X-ray fluorescence imaging for investigating a sample, and comprises an X-ray radiation source device, a detector device, and an evaluation device. The X-ray radiation source device is configured for irradiating the sample with X-ray radiation, wherein X-ray fluorescence of a first marker substance and at least one further marker substance is excited in the sample. The detector device, comprising at least one spectrally resolving X-ray detector, preferably a plurality of spectrally resolving X-ray detectors, is configured for spectrally resolved detection of the X-ray fluorescence of the first and the at least one further marker substance in the sample, wherein the X-ray fluorescence of the first and of the at least one further marker substance have different fluorescence lines in each case. The evaluation device is configured for determining spatial distributions of the first marker substance and the at least one further marker substance in the sample, from the detected X-ray fluorescence emissions. The imaging apparatus is preferably configured to carry out the method according to the first general aspect of the invention or an embodiment thereof.

According to a third general aspect of the invention, the above objective is achieved by a marker substance kit which is configured to be introduced into a sample for multi-parametric X-ray fluorescence imaging on the sample and which contains at least two marker substances which emit X-ray fluorescence upon irradiation with X-ray radiation, wherein fluorescence lines of the marker substances are different in each case. The marker substance kit is preferably provided for use in the method according to the first general aspect of the invention or an embodiment thereof, in particular for application in the sample. The marker substance kit can be provided in liquid or solid form. The composition of the marker substance kit (substances, concentrations, particle sizes) and a preferred photon energy of the X-ray radiation irradiated in can be determined by test or reference measurements and/or numerical simulations.

The spatially resolved detection of the X-ray fluorescence of the first and of the at least one further marker substance in the sample comprises detection of the X-ray fluorescence of the marker substances exclusively in at least one spatially limited region in the sample, e.g. in the region of at least one organ, and/or at least one other part in the organism, and/or a spatially resolved detection of the X-ray fluorescence of the marker substances over the entire sample.

The distributions of the first marker substance and the at least one further marker substance comprise, in each case, an assignment of quantity values, such as concentrations (or areal densities), absolute substance amounts, and/or relative prevalence of different marker substances of the marker substances, to the at least one spatially limited region and/or to location coordinates in the body. The distributions of the at least two marker substances result from the transport thereof in the sample, e.g. by means of diffusion and/or carrier fluids, such as blood, and from their biological/chemical interaction with the sample. The quantity values can be determined directly from the amplitudes of the X-ray fluorescence emissions of the marker substances, because the amplitudes are a measure for the number of detected X-ray fluorescence elements. The detected amplitudes of the X-ray fluorescence emissions are furthermore dependent on transmission, known per se, of the emitted fluorescence photons in the sample. It is possible for spatial and/or temporal distributions of the marker substances to be determined.

The spatial distributions of the marker substances in the sample in each case comprise the assignment of the quantity values of the marker substances to the at least one spatially limited region and/or to the location coordinates in the body at a specific time.

The temporal distributions of the marker substances in the sample in each case comprise a time-dependency of the assignment of the quantity values of the marker substances to the at least one spatially limited region and/or with the location coordinates in the body.

A distribution thus comprises for example average concentration values (and/or the time function thereof) in particular in organs, and/or mapping of mass values on particular location coordinates, such as a particular scan line or a particular scan surface or a particular scan volume.

The invention advantageously expands the XFI such that the specific distributions of a plurality of different marker substances, such as molecular marker elements or nanoparticles, which in each case carry different active substance molecules (active substances), such as medicaments or antibodies, or entire biological cells, in which they are contained, or which are free of active substance molecules, can be measured simultaneously and at the same sensitivity, at a minimum radiation dose, in vivo. This was ruled out, in principle, in the case of conventional XFI or PET. Compared with the conventional XFI method comprising a single marker substance, the preferably simultaneous detection of the distributions of a plurality of different marker substances (“multi-parametric X-ray fluorescence imaging”) provides additional information relating to the sample investigated, such as pharmacokinetic information and/or diagnostically evaluable information. Thus, for example the measurement of multi-parametric pharmacokinetics, and the multi-parametric tumor diagnosis, are made possible as new applications of XFI. For the first time, the invention makes it possible to determine, for example, the influence, on the kinetics, of nanoparticles, which can be used as marker substances, because at least one control group comprising non-functionalized nanoparticles is simultaneously also measured.

For example it is possible, for the first time, to determine in vivo whether a medicament is present in a sufficient concentration at a particular location in the sample, and whether another medicament that interacts therewith (e.g. has an inhibiting effect) is also present, in a significant concentration, at the same location.

A further application is that of simultaneous measurement of the pharmacokinetics of medicaments, e.g. of a new medicament and a medicament that is already approved, or an alternative medicament, or generic drugs.

According to the invention it is possible to study not only the effectiveness of medicaments, but also their interaction in the case of multi-medication, i.e. when a multiple medicaments have been administered simultaneously. The distribution of different medicaments can be determined by binding to different marker substances and detecting their distributions. It is thus possible to accurately detect where, in the sample, different medicaments having a particular concentration interact, and thus possibly interfere with one another's respective effect. This is a significant advantage for the development of medicaments.

A method for application in multi-parametric tumor diagnosis would be performed for example such that different marker substances are in each case coupled to different antibodies, in order to identify the sub-type of a tumor studied. This is a great advantage for subsequent therapy, because the optimal therapy depends on the sub-type of the tumor. In particular in those cases in which no biopsy is possible (e.g. a tumor in the respiratory center of the brain), it would be a decisive advantage for subsequent treatment if the sub-type is known.

An important feature of the invention consists in applying XFI with different marker substances, which are referred to here as the first and further marker substances. The various marker substances differ inherently, e.g. in the inside thereof, by different fluorescent elements (X-ray fluorescence elements) in each case, which result in different fluorescence lines. As a further important feature, in the non-functionalized state, i.e. without coupled active substances, the different marker substances can be identical with respect to their interaction with the sample, such that they thus have the same biological, chemical, physiological and/or physical effect on the sample, i.e. are identical for the sample. In the non-functionalized state, the marker substances are thus indistinguishable from their surroundings, in particular biologically and/or chemically.

Furthermore, the fluorescence lines of the marker substances are different to such an extent that a detector having finite spectral resolution (energy resolution) can distinguish the respective X-ray fluorescence elements in the measured X-ray spectrum of the X-ray fluorescence emissions of the marker substances. The X-ray fluorescence elements of the marker substances are preferably selected such that the K- and L-alpha/beta lines of said elements have a spectral spacing that is such that the detector device can distinguish these lines in the measured X-ray spectrum. Furthermore, the X-ray fluorescence elements of the marker substance preferably have an equal or mutually approximated fluorescence probability (effective cross section) and transmission through the sample, as well as comparable minimum background noise levels.

These features are fulfilled for the elements which are cited below, by way of example, and are preferably used, wherein typically K-alpha lines of two elements adjacent in the periodic system are superimposed, but two further not being so, and thus appearing separately in the spectrum. Since all four lines have similar energies, and are therefore absorbed similarly in the sample, all four lines can be measured at the same or a comparable degree of accuracy.

The spectrally resolved detection of the X-ray fluorescence provides an X-ray spectrum having additive superimposition of the fluorescence lines of the marker substance. The individual quantitative contributions of the fluorescence lines can be determined from the X-ray spectrum by numerical deconvolution and/or by comparison with predetermined reference measurements. The sought quantity values of the marker substances (e.g. concentrations, absolute substance amounts, and/or relative prevalence of different marker substances) result from the quantitative contributions of the fluorescence lines. Even if all types of marker substances are present in the X-ray volume, the relative prevalence thereof can be determined from the X-ray spectrum, because the X-ray fluorescence lines of all the X-ray fluorescence elements can be clearly distinguished from one another, in the spectrum.

In practice, the optimization for the sample specifically considered can be carried out by numerical simulations of the background noise and/or test measurements at different irradiation photon energies of the X-ray radiation.

According to a further preferred embodiment of the invention, the X-ray fluorescence emissions of the first and the at least one further marker substance are excited and simultaneously detected using a common excitation beam (or interrogation beam) of the X-ray radiation. Advantageously, the radiation exposure of the sample, and the duration of the method, are minimized thereby.

According to an alternative embodiment of the invention, the X-ray fluorescence emissions of the first and the at least one further marker substance are excited and detected, simultaneously or sequentially, using different excitation beams, in each case, of the X-ray radiation, which beams have different energies (irradiation photon energies). For this embodiment, the X-ray radiation source device is configured for generating multiple excitation beams of the X-ray radiation, in that for example a plurality of sources, directed onto the sample, are operated at different energies, simultaneously or sequentially (e.g. in direct succession). Advantages may result from adapting the energy of the excitation beams to the absorption of the X-ray fluorescence elements of the marker substances, in each case. The simultaneous excitation and detection have advantages with respect to the minimization of the duration of the method. The sequential excitation and detection mean that the different marker substances are excited, and the associated fluorescence is detected in a step-wise manner, preferably in direct succession. In this variant, advantages result for the direct detection of the marker substances from the X-ray spectra thereof that are recorded in succession.

A further advantage of the invention is that different types of marker substances are available, which comprise nanoparticles (or target particles) and marker molecules. The first and each further marker substance in each case comprises a plurality of nanoparticles and/or a plurality of marker molecules. Nanoparticles are particles which have a typical dimension in a range of from

2 nm to 100 nm or more, the surfaces of which are suitable or purposely prepared for coupling of ligands and/or active substance molecules. Marker molecules are single molecules or molecule aggregates which contain the X-ray fluorescence element and are suitable for coupling of active substance molecules. It is possible for all the parts of a marker substance having a particular X-ray fluorescence element to consist exclusively of nanoparticles or exclusively of marker molecules, or for a marker substance to comprise nanoparticles and marker molecules which both contain the same or different X-ray fluorescence elements.

Marker substances in the form of nanoparticles have particular advantages for the binding of active substances. Ligands, with which active substance molecules can be coupled, or which are themselves used as an active substance, are bound on the surface of the nanoparticles. The active substance molecules are located on the surface of the nanoparticles. Optionally, active substance molecules can also be arranged in the interior of the particles. In this case, the nanoparticles advantageously form active substance carriers, such as in conventional drug carrier techniques. It is thus possible for nanoparticles to be used in which different ligand molecules are bound on the surfaces, in order to provide different marker substances (such that it is possible to study which ligands dock where in the body), and at the same time the same or other active substance molecules are arranged in the inside. However, in most applications ligand molecules on the particle surface simultaneously form the active substance.

Marker substances consisting of different nanoparticles comprise, for example, a first plurality of nanoparticles (or group or type of nanoparticles) which are not functionalized, and at least one further plurality of nanoparticles to which in each case at least one predetermined medicament, to be investigated, is bound. Multi-parametric XFI with said marker substances results, with one measurement simultaneously or sequentially, in the local concentration of the non-functionalized nanoparticles and that of the functionalized nanoparticles, wherein it is possible for the difference in the concentrations to be attributed to the medicament with which the functionalized nanoparticles are coupled, because the two types of nanoparticles preferably differ, for the sample, only by the active substance or ligand molecule. In a further variant, a third type of nanoparticles can be functionalized with a second medicament. This multi-parametric XFI variant makes it possible for a basic distribution of the non-functionalized nanoparticles to be subtracted from the measured concentrations of the functionalized nanoparticles, and even for the two different medicaments to be distinguished in the process. The corresponding methods can also be implemented using marker substances which comprise marker molecules, instead of the nanoparticles.

Nanoparticles are particles which consist exclusively of one X-ray fluorescence element (optionally in a chemical compound) or of a composition of one X-ray fluorescence element (optionally in a chemical compound) and at least one further element. It is thus possible, according to a variant of the invention, for each type of nanoparticles to contain exclusively one of a plurality of X-ray fluorescence elements, optionally in a composition with a non-fluorescent element. According to an alternative variant of the invention, the first marker substance may comprise a first type of nanoparticles, which primarily contain a first X-ray fluorescence element, and the at least one further marker substance may comprise at least one further type of nanoparticles, which in each case primarily contain at least one further X-ray fluorescence element. Thus, nanoparticles can in each case contain at least two X-ray fluorescence elements, one X-ray fluorescence element of which is decisive for the fluorescence line to be detected in each case. This may have advantages for the design of the nanoparticles, e.g. in the case of the core-shell structure mentioned below.

According to a further advantageous embodiment of the invention, the first type of nanoparticles can carry a first type of active substance molecules which have a chemical and/or physical interaction with the sample, while each further type of nanoparticles in each case carries another type of active substance molecules, which may have a chemical and/or physical interaction with the sample that deviates from the first type, or does not carry any active substance molecules. Particularly preferably, each type of nanoparticles carries exclusively one specific type of active substance molecules. This advantageously increases the conclusiveness of the XFI. Advantageously, the nanoparticles thus offer a high degree of flexibility in the case of adaptation to a specific XFI investigation task.

According to a further preferred embodiment of the invention, at least one type of nanoparticles may have a core-shell structure, comprising a particle core and a particle cover layer (hybrid nanoparticle). The core-shell structure advantageously allows for separation of two functions of the nanoparticles, firstly with respect to the X-ray fluorescence emission, and secondly with respect to the interaction with the surroundings. Thus, the particle core can be produced from the X-ray fluorescence element having the desired fluorescence line of the respective type of nanoparticles, while the particle cover layer is produced from different material from the core, and forms a surface for coupling ligands and/or active substance molecules, and for providing a predetermined biological and/or chemical interaction with the sample. The particle cover layer can be produced from a fluorescent or a non-fluorescent element.

According to preferred variants of the invention, the particle cover layer is formed of a metal, in particular gold, or a non-metal material, in particular a polymer or a liposome material or a micelle material. Since the coupling chemistry of active substances with nanoparticles has hitherto been studied in particular using metal nanoparticles, in particular gold nanoparticles, nanoparticles having the core-shell structure preferably have an X-ray fluorescence element, in the particle core, which has an atomic number comparable to that of gold (such that the X-ray fluorescence is in a comparably high energy range, in order to measure these photons at a high degree of sensitivity, outside of the sample), and the particle core is preferably covered by a metal layer, in particular gold layer, for the coupling of the active substances (ligands). The thickness of the particle cover layer is preferably e.g. ¼ of the particle diameter, or less. Therefore, the volume fraction of the gold compared with the other X-ray fluorescence element is negligible, such that the XFI signal appears as though there were only the X-ray fluorescence element of the particle core. An alternative possibility is that of producing nanoparticles from the various X-ray fluorescence elements, and using, instead of a metal, a polymer for the particle cover layer, as is described for example in [5]. The corresponding ligands, i.e. for example the medicaments or antibodies, can then be bound to said polymer particle cover layers. The polymer layer nanoparticles can also be combined with suitable inner X-ray fluorescence elements in order, depending on the specific application conditions (e.g. size and amount of the medicament studied), to implement a multi-parametric XFI in which all the nanoparticles used have a similar sensitivity (ratio of signal strength to statistical noise of the relevant background).

Particularly preferably, all the nanoparticles of the various marker substances have the core-shell structure, wherein the particle cores of the various marker substances are made from different X-ray fluorescence elements, and the particle cover layers of all the marker substances are made from the same element, to which at least one of active substance molecules and ligand molecules may be bound. Optionally, the particles of one marker substance of a plurality of marker substances can be formed entirely of the element from which the particle cover layers of the remaining marker substances are formed.

According to a preferred design, the nanoparticles are externally indistinguishable, while they comprise different elements on the inside, wherein they preferably have the same size. The nanoparticles having the core-shell structure preferably have the particle cover layers on the outside, which are formed of an identical material and are preferably identical. They therefore cannot be distinguished by the sample, in particular by the body of a studied biological organism, unless they are differently functionalized. In the interior, said nanoparticles comprise different elements, the X-ray fluorescence energies of which are different. It is thus possible to distinguish the different nanoparticle types from one another, in a measured XFI spectrum, and to simultaneously determine their respective concentrations, while, with the exception of the functionalization, they are indistinguishable by the sample.

According to particularly preferred embodiments of the invention, the nanoparticles in each case contain, as X-ray fluorescence elements, in particular in the particle core, iridium or platinum or gold or bismuth. On account of their similar but distinguishable X-ray fluorescence energies, these elements can advantageously be detected at a comparable level of sensitivity. As a result, it is even possible for up to four different medicaments, (immune) cell types, and/or sub-type-specific antibodies, e.g. in the body of a biological organism, to be tracked simultaneously. According to alternative variants, the nanoparticles can in each case contain different X-ray contrast agent molecules. Advantageously, X-ray contrast agent molecules, such as iodine or barium or gadolinium, are widely available in practice and well studied with respect to their absorption behavior. Iodine and barium are in particular preferred for imaging on small animals. Nanoparticles made of silver, palladium indium, cadmium or iodine are also advantageous for imaging on small animals.

If, according to further preferred embodiments of the invention, different nanoparticles, i.e. the different marker substances having different X-ray fluorescence elements, are of a different nanoparticle size in each case, a further degree of freedom is provided in the multi-parametric XFI. It is advantageously possible, as a result, for the conclusiveness of XFI to be further increased, and/or for the behavior of nanoparticles in the sample to be investigated. For example, it is known from practice that nanoparticles of different sizes can have different kinetics. For example distributions of nanoparticles comprising up to four different sizes could be detected by means of X-ray fluorescence. In this case, typical sizes are selected so as to be in the diameter intervals of 2 nm to 5 nm, 6 nm to 10 nm, 11 nm to 20 nm, and 21 nm to 50 nm. The nanoparticles of different sizes are preferably of the same surface types, such that only the size, and, depending on the size, the corresponding X-ray fluorescence element, are varied.

Alternatively or in addition, the nanoparticles of different marker substances can also differ with respect to the supply into the sample. For example it is possible to investigate, using the multi-parametric XFI, the effect of different administration pathways, e.g. oral and intravenous supply of the nanoparticles, on the distribution of the marker substances in the sample.

Marker substances in the form of marker molecules have particular advantages for the transport in the sample. Marker molecules are significantly smaller than nanoparticles, and therefore their transport in the sample is more similar to the molecular mass transfer in samples, in particular in biological organisms. Preferably, the first marker substance comprises a first type of marker molecules, which contain a first X-ray fluorescence element, and every further marker substance in each case comprises a further type of marker molecules, which in each case contain at least one further X-ray fluorescence element.

If, according to a further advantageous embodiment of the invention, the first marker substance comprises nanoparticles which contain a first X-ray fluorescence element, and the at least one further marker substance comprises marker molecules which in each case contain at least one further X-ray fluorescence element, advantages for new applications of XFI are achieved. For example it is possible for a plurality of, e.g. four, marker substances having different nanoparticles to be combined with one or more further marker substances being made of marker molecules in the form of contrast agent molecules.

It is possible for example to couple three different types of immune cells with three different nanoparticles, and to additionally study a medicament to which a fluorescent marker molecule is bound. The marking of the different immune cell types with different nanoparticles in each case is performed e.g. by prior removal of the cells and subsequent loading with the nanoparticles and introduction into the sample, or by using nanoparticles which are functionalized such that they in each case bind, in vivo, only to specific, different immune cell types. The nanoparticles for the immune cells should be indistinguishable for the cells and the sample, while the medicament differs from the immune cells. In other words, in this case the medicament does not necessarily have to also be bound to a nanoparticle. This application may have particular advantages in the study of immunobased diseases, such as Crohn's disease. The distributions of different immune cell types and the distribution of the medicament could be measured simultaneously, in order to thereby study, in vivo, the effectiveness of the medicament. In this case, the effect may be that the medicament changes the prevalences of the immune cell types in the inflammation region, for example immune cell types which ease inflammation then appearing more frequently.

A further advantageous application of the invention, in which both nanoparticles and marker molecules are used as different marker substances in each case, is what are known as drug carriers. It is possible for example to use two different marker substances with different nanoparticles in each case, of which one group of nanoparticles is non-functionalized, and the other group of nanoparticles is functionalized with a predetermined ligand which is intended to dock onto a target structure in the sample. In this case, both nanoparticles can serve as a drug carrier, i.e. have inside the actual medicament to which molecule-based markers are now bound. It is possible to determine, by means of the multi-parametric XFI, where and optionally when the nanoparticles, as drug carriers, release their load. An early release would be identifiable for example if the medicament distribution does not correspond to the distribution of the nanoparticles. The comparison of the distributions of the non-functionalized nanoparticles with those carrying the ligands can show how specifically the ligands find their target location. If both distributions are identical, it would be found that the drug carrier arrives at the target location rather coincidentally and not in a targeted manner, which is what should actually be achieved by the ligands.

The marker molecules are preferably bound to active substance molecules, wherein the marker molecules in each case comprise one of the first and at least one further X-ray fluorescence element. The X-ray fluorescence elements particularly preferably include moderately heavy elements from zirconium to cerium, for which the background noise level can be significantly reduced, or heavy elements such as iridium, platinum, gold and bismuth. These two groups of elements have advantages on account of their similar and already well-studied fluorescence properties, and can be easily coupled to medicament or ligand molecules.

According to the invention, spatial and/or temporal distributions of the marker substances may be determined. In this case, a preferred embodiment of the invention in which the measurement is both spatially and temporally resolved, and a time function of the spatial distributions of the first marker substance and the at least one further marker substance in the sample is determined, is particularly advantageous. This embodiment has a particularly high degree of conclusiveness regarding the transport of the marker substances in the sample, from the introduction into the sample to the binding within the sample.

According to a further feature which is preferably implemented, the irradiation photon energy of the X-ray radiation for exciting the marker substances, and X-ray fluorescence properties of the marker substances, are preferably selected such that the irradiation photon energy of the X-ray radiation is above the absorption edges of the X-ray fluorescence elements of all the marker substances, and the X-ray fluorescence elements of all the marker substances exhibit fluorescence probabilities, attenuations of the X-ray fluorescence in the sample, and signal-to-background noise levels in the sample which are equal or are similar to such an extent that the detection of the X-ray fluorescence at the same concentration results in comparable signal strengths. This feature advantageously makes it possible to more easily determine relative prevalences of different marker substances directly from the detected X-ray spectra. It is possible, for example, for one of a plurality of marker substances to have a significantly lower concentration than the other marker substances, because, for example, the ligands on these nanoparticles couple to target structures in the sample only with difficulty. This behavior can be observed in the case of detection of the X-ray fluorescence at a comparable sensitivity, directly from the detected X-ray spectra. If all the marker substances have the same or a similar areal density, their signals will have comparable intensities.

According to a preferred application of the invention, the sample investigated is a human or animal subject, or a body part thereof. The marker substances are introduced into the subject in advance, e.g. by oral or other administration or injection. The first and the at least one further marker substance can in each case be introduced into the sample in different ways. A preparation step comprising introduction of a marker substance by injection into the body of the subject is not part of the invention.

According to a further advantageous application of the invention, the active substances comprise biological cells, the at least one of the first and the at least one further marker substance being coupled to the biological cells, and the determination of the distribution of the first marker substance and the at least one further marker substance comprising a detection of transport of the biological cells through the sample. It is advantageously possible, as a result, for example for the transport of different immune cells through the sample to be determined.

Further details and advantages of the invention will be described in the following, with reference to the accompanying drawings, in which:

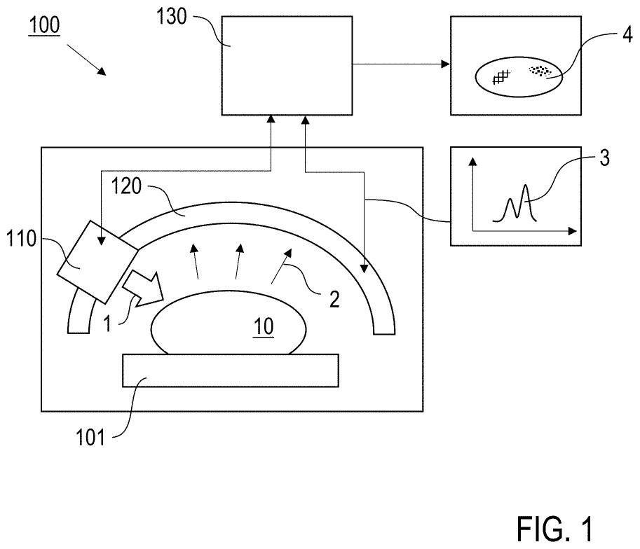

FIG. 1: is a schematic view of an imaging apparatus for X-ray fluorescence imaging according to an embodiment of the invention;

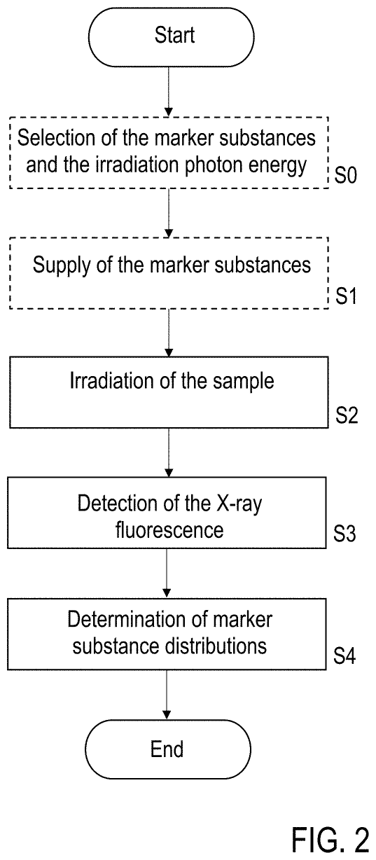

FIG. 2: is a flow diagram of a method for X-ray fluorescence imaging according to embodiments of the invention;

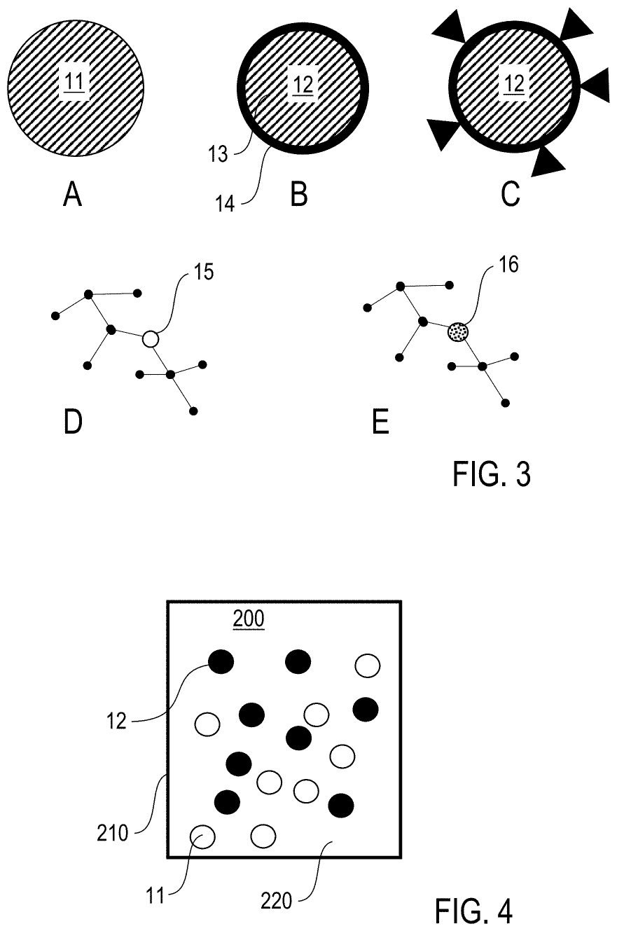

FIG. 3: shows examples of marker substances which can be used in the method according to the invention for X-ray fluorescence imaging;

FIG. 4: is a schematic view of a marker substance kit according to an embodiment of the invention;

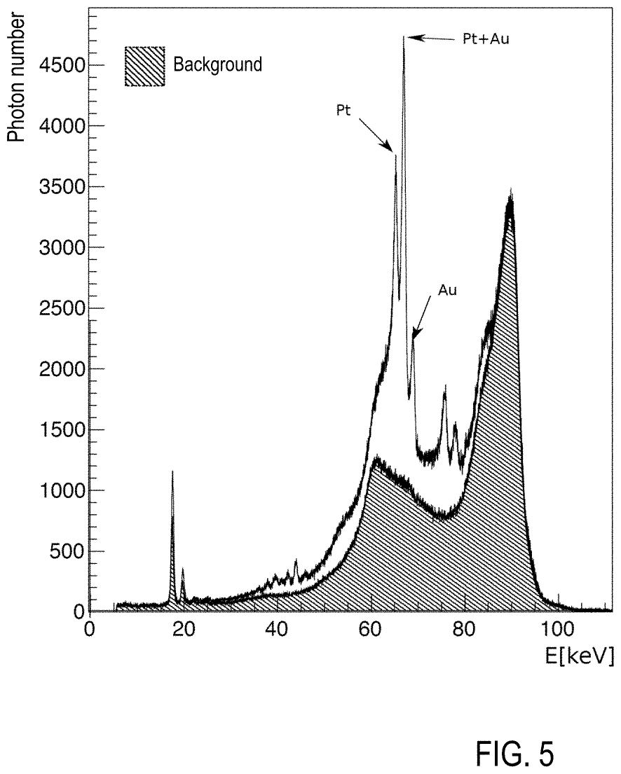

FIG. 5: shows a measured X-ray spectrum for illustrating X-ray fluorescence emissions of two different X-ray fluorescence elements in biological cells;

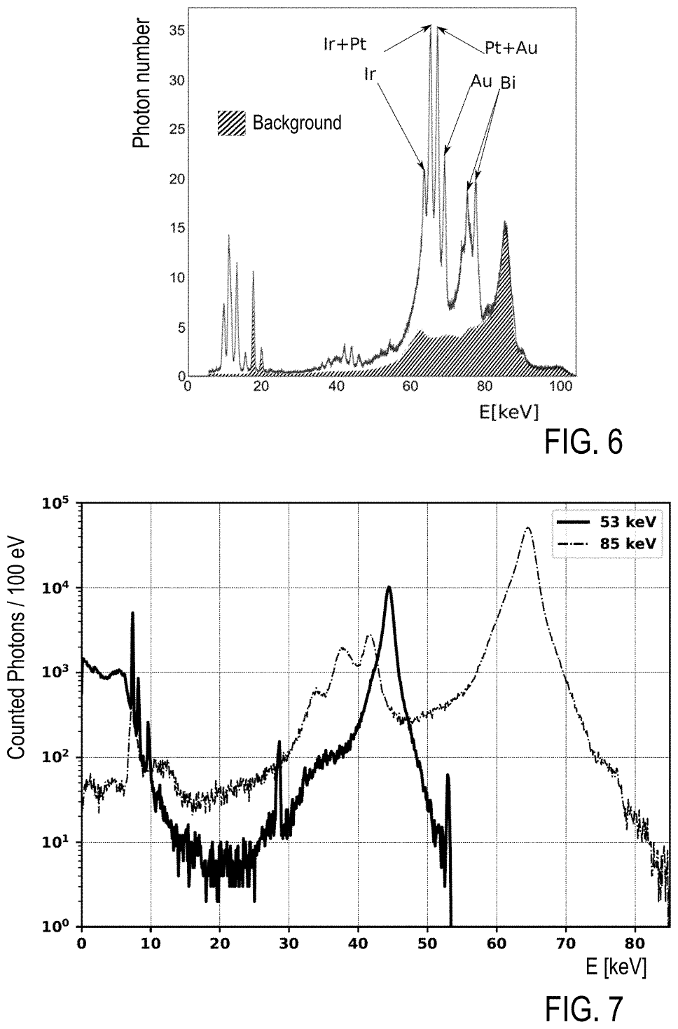

FIG. 6: shows a measured X-ray spectrum for illustrating X-ray fluorescence emissions of four different X-ray fluorescence elements in a sample; and

FIG. 7: shows simulated X-ray spectra for illustrating X-ray fluorescence emissions of X-ray fluorescence elements in a sample for two different irradiation energies.

Features of preferred embodiments of the invention are described in the following, with reference, by way of example, to XFI on a human subject, with particular X-ray fluorescence elements. It is emphasized that the implementation of the invention in practice is not limited to the stated examples, but rather is possible, in a corresponding manner, using other samples, such as portions of a human subject, synthetic biological objects, animal subjects, or portions thereof. Embodiments of the invention are described in the following, in particular with reference to important features of the configuration of the marker substances, the execution of the XFI, and the structure of the imaging apparatus. Further features of the imaging apparatus can be implemented for example as described in [1]. [1] is incorporated by reference into the present disclosure, with respect to the structure and the function of the imaging apparatus. Details of the functionalization of nanoparticles, the coupling of nanoparticles with active substances, the selection of ligands, the coupling of marker molecules with active substances, the spectrally and spatially resolved detection of X-ray fluorescence, and the analysis of superimposed spectra composed of a plurality of fluorescence lines are not described, insofar as they are known per se from the prior art. The concentrations of the marker substances can be selected as is known per se from conventional XFI.

FIG. 1 schematically shows features of embodiments of an imaging apparatus 100 for X-ray fluorescence imaging for the study of a sample 10, such as a human subject, which is arranged on a sample holder 101, such as a bed. FIG. 2 schematically shows the steps of the method according to the invention using the imaging apparatus 100, comprising the supply of the marker substances (S1), the irradiation of the sample with X-ray radiation (S2), the detection of the X-ray fluorescence (S3), and the determination of marker substance distributions from the detected X-ray fluorescence (S4).

In addition, FIG. 2 shows a step S0, which comprises the selection of the marker substances and the irradiation photon energy. Is it sufficient for step S0 to be performed once, and separately from the implementation of the method according to the invention, for a considered sample or group of samples having the same scattering properties, for example for small animals of a particular species and size. Alternatively, step S0 can be provided as part of each execution of the method according to the invention.

The selection is made for example under the following considerations, on the basis of the fluorescence spectra shown by way of example in FIG. 7. In detail, FIG. 7 shows a direct comparison of two simulated spectra following excitation with monochromatic X-rays, firstly for an irradiation energy of for example 85 keV, and secondly for an irradiation energy of for example 53 keV. In the first case, it would be possible for example for gold nanoparticles to be excited (K edge at approximately 81 keV) which, however, have fluorescence lines in the region of the high peak around approximately 65 keV, said peak originating from just once-scattered photons. In contrast thereto, the 53 keV spectrum exhibits a pronounced background minimum at those fluorescence lines of moderately heavy elements which are in the range of between approximately 15 and 28 keV, because, for this, incoming photons usually have to scatter 5 times or more. Although the 85 keV spectrum also exhibits a minimum in the same energy range, this is higher, and the high irradiation energy would mean smaller yields for the fluorescence of moderately heavy elements.

Thus, for a marker kit of moderately heavy elements, the lower irradiation energy is more efficient, and for heavy elements of gold the higher energy is preferred. A variation of the detector position relative to the beam direction (in this case 150°) makes it possible for the minimum in the background range to be expanded slightly more, but the decisive parameter for the level of the minimum is the irradiation energy.

In the specific example of XFI on mice, for a first marker substance iodine can be selected as the X-ray fluorescence element, and 53 keV as the irradiation photon energy (see FIG. 7). The irradiation photon energy of 53 keV is significantly above the iodine edge of 33 keV. Incoming photons, which, after multiple scattering, fall into the energy range of the X-ray fluorescence of iodine (approx. 29 keV), would thus have to perform a plurality of successive Compton scatterings, in particular approximately 5 Compton scatterings. With each further Compton scattering after the preceding scattering, the overall probability reduces, such that the background is minimized. At the same time, upon excitation at 53 keV, iodine has a sufficiently high fluorescence probability. In this example, an element that is closely adjacent to iodine in the PTE, such as indium, is provided as a further marker substance, which is excited at the same irradiation photon energy of 53 keV.

The selection of the marker substances and the irradiation photon energy is performed for example by simulation of the scattering behavior of the sample and/or test, according to the above-mentioned optimization, such that the irradiation photon energy of the X-ray radiation is at a distance from a highest absorption edge of all the marker substances in the sample, at which, at the same time, the background noise levels of the marker substances are minimal and the effective cross sections are similar.

In a further example of XFI on larger samples, for example gold and an element closely adjacent to gold in the PTE, such as platinum, would be used as the X-ray fluorescence element, and an irradiation photon energy of the X-ray radiation of for example 85 keV would be used.

Step S1 comprises for example oral delivery and/or an injection of the marker substances and can, if an intervention into a biological body is provided, be considered to be no part of the invention.

The imaging apparatus 100 comprises an X-ray radiation source device 110, which is configured for irradiating the sample 10 with X-ray radiation 1 (step S2), and emits for example a photon energy of 50 keV or 100 keV. The X-ray radiation source device 110 is preferably a compact laser-based Thomson source (X-ray radiation source which generates X-ray radiation based on the Thomson scattering of laser light at relativistic electrons), as is described for example in [8], but can also comprise a synchrotron source or in general an X-ray source, e.g. an X-ray tube, which generates X-ray radiation having a sufficiently low divergence and high intensity, in particular in the energy range above the K edge of the X-ray fluorescence elements of the marker substances, and should be as monochromatic as possible, in order for the above-described optimization of the irradiation energy to be improved.

The X-ray radiation 1 may be generated in the shape of a parallel radiation beam having a diameter which covers the entire cross section, to be investigated, of the sample 10. In this case, all the regions of the sample are irradiated simultaneously, and the marker substances present therein are excited to X-ray fluorescence 2. Alternatively, the X-ray radiation 1 can be generated as a pencil beam, in particular having a smaller diameter than the cross section of the sample, transversely to the beam direction, and can be moved (scanned) relative to the sample 10 by means of X-ray optics (not shown). In this case, the regions of the sample are irradiated (“scanned”) in succession and the marker substances present therein are excited to X-ray fluorescence 2. Since the scanning movement of a pencil beam over the sample 10 can take place within a scanning duration which is negligible compared with typical transport times of marker substances in the sample 10, a snapshot of the X-ray fluorescence 2 is also acquired effectively in the case of scanning of the X-ray radiation 1.

The imaging apparatus 100 further comprises a detector device 120 which is arranged for spectrally and spatially resolved detection of the X-ray fluorescence 2 of marker substances in the sample 10 (step S3). The detector device 120 comprises a plurality of detector elements (not shown) which in each case acquire an X-ray spectrum of the X-ray fluorescence 2. The corresponding solid angle range, which covers a predetermined geometric portion in the sample 10, can be restricted by collimators of the individual detector elements or on groups of detector elements. The detector device 120 is constructed for example as is described in [1]. A collimator may be arranged between the detector device and the sample, which collimator can reduce scattered radiation, as described in [7].

In a manner deviating from FIG. 1, the detector device 120 may be provided with just one detector element, which is movable relative to the sample 10 and is arranged for spectrally resolved detection of the X-ray fluorescence 2 of marker substances in the sample 10. The sample 10 can be sampled (scanned) by means of the movable detector element, in order to acquire a spatial distribution of the marker substances, if a collimator cuts out only certain regions of the sample in the solid angle range of the detector element. According to a further alternative, a single detector element can be arranged so as to be fixed in position relative to the sample 10, and arranged for spectrally resolved detection of the X-ray fluorescence 2 of marker substances in a particular portion of the sample 10. In this case, too, the X-ray fluorescence 2 is acquired in a manner spatially restricted to a defined part of the sample 10, e.g. an organ, if a collimator is used.

Alternatively, the marker substances can be located without collimators, by scanning of the X-ray beam 1, e.g. as is described in [7].

The sample 10 contains at least two marker substances each having different X-ray fluorescence elements, which are excited to X-ray fluorescence 2, by the X-ray radiation 1. The detector device 120 delivers output signals in the form of X-ray spectra, which are in each case associated with pre-determined portions (geometric positions) in the sample 10 and which contain a superimposition of the fluorescence lines 3 of the X-ray fluorescence elements (see schematic graph of a spectrum in FIG. 1, and example measurement in FIGS. 5 and 6).

The imaging apparatus 100 furthermore comprises an evaluation device 130 for receiving the output signals (spatially resolved X-ray spectra) of the detector device 120, and for determining spatial distributions 4 of the marker substances in the sample 10 from the detected X-ray fluorescence 2 (step S4). The evaluation device 130 comprises for example a computer device which is coupled to the detector device 120. The evaluation device 130 is configured for executing a computer program, by means of which the intensities of the fluorescence lines at the geometric positions in the sample 10, and from these the sought distributions 4 of the marker substances, are determined from the output signal of the detector device 120, preferably taking into account a previously determined background spectrum.

The distributions 4 of the marker substances (see schematic illustration in FIG. 1) are output for example as an image (map) or tabular values. Upon acquisition of a temporal distribution of the marker substances, it is possible for a sequence of moving images, e.g. a video sequence, to be output, which represents the movement of the marker substances in the sample 10 and/or an accumulation of at least ones of the marker substances in a portion of the sample 10, such as an organ.

The computer device can optionally additionally be provided as a controller of the imaging apparatus 100, in particular for controlling and/or monitoring the X-ray radiation source device 110 and/or the detector device 120.

The sample 10 contains a first and at least one further marker substance, which differ in terms of their fluorescence lines 3 and will be described in the following, by way of example, with reference to FIG. 3. FIGS. 3A to 3C show a first type of marker substances in the form of nanoparticles 11, 12, while FIGS. 3D to 3E show a second type of marker substances in the form of marker molecules 14, 15. The nanoparticles 11, 12 may be spherical in shape (as is shown by way of example), or may be of a different shape, e.g. an angular shape having a plurality of side faces, and/or a rod shape.

According to FIG. 3A, a nanoparticle 11 may be produced from a single X-ray fluorescence element, in particular may consist entirely of the X-ray fluorescence element, such as gold or platinum, and have a diameter of for example 10 nm. According to FIG. 3B, a nanoparticle 12 may have a core-shell structure comprising a particle core 13 and a particle cover layer 14. Just like the nanoparticles 11 according to FIG. 3A, the particle core 13 may be produced from a single X-ray fluorescence element, in particular may consist entirely of the X-ray fluorescence element, such as platinum. The particle cover layer 14 consists of a different material from the particle core 13, e.g. of gold or a polymer (see [5]). The particle cover layer 14 has a thickness of e.g. 2 nm. According to FIG. 3C, a nanoparticle 12 having a core-shell structure and/or a particle cover layer can be functionalized, i.e. provided on the surface thereof with ligand and/or active substance molecules. The ligand and/or active substance molecules are illustrated schematically in FIG. 3C by means of triangles, and can in particular comprise entire biological cells.

Each marker substance comprises a plurality of nanoparticles 11, 12, the substance amount of which is selected depending on the desired concentration in the sample and the desired sensitivity in the measurement of the X-ray spectra using the detector device 120. The selection of the X-ray fluorescence elements of the nanoparticles, and optionally the functionalization of the nanoparticles, are implemented taking into account the following considerations.

In the Periodic Table of the Elements (PTE), elements that are located close to one another have physically very similar properties with respect to the production probability and the attenuation of X-ray fluorescence. At a given energy of the X-ray photons radiated in, the first variable depends only on the element. Accordingly, the X-ray fluorescence elements of the nanoparticles are selected such that they are directly adjacent in the PTE or are so close together that the X-ray fluorescence of all the X-ray fluorescence elements can be measured at a comparable sensitivity. X-ray fluorescence elements in different nanoparticles comprise for example at least two of iridium, platinum, gold and bismuth, because the four heavy elements are close to one another in the PTE (Ir, Pt and gold are indeed directly adjacent). Thus, their behavior is very similar, and all four can be used simultaneously for XFI. A further expedient variant would be moderately heavy X-ray fluorescence elements from zirconium to cerium. In contrast, it would be unfavorable to form nanoparticles of two different marker substances for example such that some nanoparticles consist of gold, on the inside, and other nanoparticles consist of an iodine compound. The two elements gold and iodine are so far apart from one another in the PTE that they can emit X-ray fluorescence simultaneously only if the irradiation energy is above what is known as the gold edge—if the energy were below said edge, no gold X-ray fluorescence would be excited. However, since the iodine edge is far distant from this, the probability of an iodine fluorescence also being generated reduces significantly. In addition, there is also the problem of the background in XFI—the (multiple) Compton scattering can lead to a strong background in the X-ray spectrum in the signal region of the actual fluorescence lines (see [1] and [7]), and would be significantly higher for gold than for iodine, such that overall the two elements could not be measured at a similar level of sensitivity.

It is advantageous if the sample, in particular the body of the subject, cannot distinguish the gold and platinum nanoparticles, because both are of the same size, have the same surface (e.g. an identical polymer particle cover layer), and are of equal or very similar masses. If, however, for example only the platinum nanoparticle, comprising a gold particle cover layer thereon, is functionalized, but the gold nanoparticle remains non-functionalized, and both are introduced into the sample, measured differences in the local concentrations can be traced back to the action of the ligands, because the two nanoparticle types are otherwise indistinguishable by the body. Only if the ligands bond purposely, in the body, will the local concentration at the binding site of the platinum nanoparticles be higher than that of the non-functionalized gold nanoparticles, which thus serve as the reference concentration. These local concentration differences can advantageously be measured by means of the XFI according to the invention. For this purpose, it is particularly advantageous for the measuring sensitivities of both internal X-ray fluorescence elements of the nanoparticles to be sufficiently high, and preferably equal or very similar (possible differences with respect to the evaluation of the detector output signals are negligible).

A further variant of the use of nanoparticles is possible such that these do not contain any heavy elements on the inside, but rather lighter molecules comprising X-ray fluorescence elements, for example the two contrast agents mentioned below for computerized tomography (CT), and bear a polymer shell as the particle cover layer.

FIGS. 3E and 3D relate to variants of the invention in which active substances, such as medicament molecules, are directly connected to marker molecules 15, 16, such as smaller complexes comprising X-ray fluorescence elements, such as a triiodobenzene ring or a ring of barium atoms. Triiodobenzene and barium are advantageously available CT contrast agents, wherein the two elements, iodine and barium, are arranged close together in the PTE. The multi-parametric XFI comprising marker molecules thus uses X-ray fluorescence element complexes comprising different X-ray fluorescence elements, to which for example different medicament molecules can be bound. The marker molecules preferably have a chemically equal or very similar effect on the sample, such that they do not influence the kinetics of the medicaments in the sample, or only in a manner that is insignificant for the measurement.

Regarding the use of different CT contrast agents as marker substances, it is noted that the conventional CT methods would not allow for multi-parametric measurement, because the measuring difference in the different absorptions of the contrast agents would be far too small for these to be measurable simultaneously. An important advantage of the invention compared with CT is that the XFI is a spectroscopic method where every element produces its own characteristic lines, and is not purely an absorption method.

FIG. 4 schematically shows, by way of example, a marker substance kit 200 for introducing marker substances into a sample for X-ray fluorescence imaging according to the invention. The marker substance kit 200 comprises a container 210, such as a flexible pouch, which is filled with a marker substance suspension 220. The marker substance suspension 220 comprises a physiological fluid, such as a physiological salt solution, in which nanoparticles 11, 12 comprising different X-ray fluorescence elements are arranged. The design of the nanoparticles 11, 12, the volume of the container 210, and the concentration of the nanoparticles 11, 12 in the marker substance suspension 220 are selected depending on the specific XFI application. In order to use the marker substance kit 200, the container 210 is connected to a blood vessel of a subject, via a line and an injection needle, and the marker substance suspension 220 comprising the nanoparticles 11, 12, is conducted into the blood vessel.

Alternatively, the marker substance kit 200 according to FIG. 4 can be provided for oral ingestion. According to a further alternative, a marker substance kit can be provided in dry form, e.g. in the form of a tablet, comprising the nanoparticles 11, 12 and a physiological binder.

With test measurements by the inventors, biological cells were provided with both gold and platinum nanoparticles in predetermined concentrations, and arranged in a reagent vessel (Eppendorf vessel) having a diameter of 6 mm. The reagent vessel was then pushed into a piece of animal flesh which was of a similar size to a mouse. The sample, comprising the flesh having the inserted reagent vessel, was irradiated with monochromatic X-ray radiation by the German Electron Synchrotron (DESY) (DESY Hamburg). In the case of further test measurements, four different fluorescent elements were arranged in a reagent vessel and irradiated with X-ray radiation from DESY synchrotron. The test measurements, which were directed to the distinguishable nature and quantitative evaluability of the measured X-ray fluorescence, yielded the results shown in FIGS. 5 and 6. The corresponding measurements with spatial resolution can for example also be performed using the technique described in [1].

According to FIG. 5, the gold and platinum fluorescence lines, detected simultaneously at a high degree of sensitivity, can be clearly distinguished. The evaluation of the superimposed fluorescence lines, comprising a numerical deconvolution for determining the individual intensities of the fluorescence lines, resulted, taking account of predetermined reference or calibration data, in the concentrations of the gold and platinum nanoparticles.

FIG. 6 shows a measured X-ray spectrum in the case of the reagent vessel, in which the four elements iridium, platinum, gold and bismuth were in solution, in predetermined concentrations. It is possible to clearly identify the four elements in the spectrum. The respective concentrations, which corresponded very well with those used, could be determined from all the fluorescence lines present.

FIGS. 5 and 6 in each case also show a background spectrum, which was measured when the reagent vessels contained only water. The measurement of the background spectrum illustrates that knowledge of the background is important for the quantitative evaluation of the individual fluorescence lines in the spectrum, in order to be able to conclude the number of corresponding fluorescence photons. The background can be measured specifically upon each use, or can be determined using reference or calibration data. In particular, the background can be selected so as to be approximately the same for all marker elements, and so as to be equally minimal for all, as far as possible.

The background curve can also be taken into account when selecting the X-ray fluorescence elements. If the absorption of the fluorescence photons of an element is too strong, such that it can barely be distinguished from the background in the spectrum at the location of the fluorescence energy, this element would be unusable.

The features of the invention disclosed in the above description, the drawings, and the claims, can be of significance both individually and in combination or sub-combination for implementing the invention in the various embodiments thereof.

1. A method for multi-parametric X-ray fluorescence imaging on a sample which comprises at least a part of a body of a biological organism and contains a first marker substance and at least one further marker substance, wherein at least one of the first and the at least one further marker substance is coupled to at least one of active substance molecules and ligand molecules which are provided for a specific interaction with the sample, comprising the steps of:
irradiation of the sample with X-ray radiation, wherein X-ray fluorescence of the first marker substance and of the at least one further marker substance is excited by the X-ray radiation, and fluorescence lines of the X-ray fluorescence, of the first and the at least one further marker substance are different,
spatially resolved detection of the X-ray fluorescence, comprising a spectrally resolved detection of the X-ray fluorescence of the first and the at least one further marker substance, and
determination of a distribution of the first marker substance in the sample and in addition at least one distribution of the at least one further marker substance from the detected X-ray fluorescence, wherein
the first and the at least one further marker substance exhibit fluorescence probabilities, attenuations of the X-ray fluorescence in the sample, and background noise levels on account of scattering in the sample which are equal or are similar to such an extent that the detection of the X-ray fluorescence at the same concentration of the marker substances results in comparable statistical significance levels, and
the irradiation photon energy of the X-ray radiation is above the absorption edges of all the marker substances.

2. The method according to claim 1, wherein
the statistical significance levels of the marker substances are maximized in that the irradiation photon energy of the X-ray radiation is selected so as to be of a distance above the highest absorption edge of the marker substances in the sample, such that the background noise levels of the marker substances are minimal and equal or approximately equal, and at the same time the fluorescence probabilities and through the sample are equal or approximately equal, and maximum.

3. The method according to claim 1, wherein
the X-ray fluorescence of the first and the at least one further marker substance is excited and simultaneously detected using a common excitation beam of the X-ray radiation.

4. The method according to claim 1, wherein
the X-ray fluorescence of the first and the at least one further marker substance is excited and simultaneously or sequentially detected using different excitation beams of the X-ray radiation which exhibit different energies.

5. The method according to claim 1, wherein
the first and the at least one further marker substance in each case comprise nanoparticles and marker molecules.

6. The method according to claim 5, wherein
the first or the at least one further marker substance in each case comprise nanoparticles, and
the nanoparticles of the first and the at least one further marker substance comprises surfaces which are indistinguishable for the sample, wherein the nanoparticles comprise different elements on the inside.

7. The method according to claim 5, wherein
the first marker substance comprises a first type of nanoparticles which primarily contain a first X-ray fluorescence element, and
the at least one further marker substance comprises at least one further type of nanoparticles which in each case primarily contain at least one further X-ray fluorescence element.

8. The method according to claim 7, wherein
each type of nanoparticles contains exclusively one of the first and the at least one further X-ray fluorescence element.

9. The method according to claim 5, wherein
the first type of nanoparticles carries a first type of at least one of active substance molecules and ligand molecules which are provided for at least one of a chemical and a physical interaction with the sample, and
the at least one further type of nanoparticles in each case carries different types of at least one of active substance molecules and ligand molecules which are provided for at least one of a chemical and a physical interaction with the sample, or does not carry any active substance molecules or any ligand molecules.

10. The method according to claim 9, wherein
each type of nanoparticles carries exclusively one specific type of at least one of active substance molecules and ligand molecules.

11. The method according to claim 5, wherein
at least one of the first and the at least one further type of nanoparticles has a core-shell structure comprising a particle core and a particle cover layer.

12. The method according to claim 11, wherein
all the nanoparticles are of the core-shell structure.

13. The method according to claim 11, wherein
the particle cover layer comprises a metal.

14. The method according to claim 11, wherein
the particle cover layers of all the nanoparticles are produced from the same material, to which at least one of active substance molecules and ligand molecules can bind.

15. The method according to claim 5, wherein
the nanoparticles in each case contain iridium, platinum, gold, bismuth, silver, iodine, palladium, cadmium or indium.

16. The method according to claim 5, wherein
the nanoparticles in each case contain different X-ray contrast agent molecules.

17. The method according to claim 5, wherein
each type of nanoparticles is of a different nanoparticle size.

18. The method according to claim 5, wherein
the first marker substance comprises a first type of marker molecules which contain a first X-ray fluorescence element, and
the at least one further marker substance comprises at least one further type of marker molecules which in each case contain at least one further X-ray fluorescence element.

19. The method according to claim 5, wherein
the first marker substance comprises nanoparticles which contain a first X-ray fluorescence element, and
the at least one further marker substance comprises marker molecules which in each case contain at least one further X-ray fluorescence element.

20. The method according to claim 18, wherein
the marker molecules are bound to at least one of active substance and ligand molecules, the marker molecules in each case comprising one of the first and at least one further X-ray fluorescence element.

21. The method according to claim 18, wherein
the X-ray fluorescence elements comprise silver, indium, palladium, cadmium, iodine or barium.

22. The method according to claim 1, wherein
a time function of spatial distributions of the first marker substance and the at least one further marker substance in the sample is determined.

23. The method according to claim 1, wherein
the active substances comprise biological cells, and the at least one of the first and the at least one further marker substance is coupled to the biological cells, and
the determination of the distribution of the first marker substance and the at least one further marker substance comprises a detection of transport of the biological cells through the sample.

24. The method according to claim 1, wherein
the first and the at least one further marker substance have in each case been introduced into the sample in different ways.

25. The method according to claim 1, wherein
the first and the at least one further marker substance are formed such that they have the same effect for the sample, without coupled active substance molecules or ligand molecules.

26. An imaging apparatus which is configured for multi-parametric X-ray fluorescence imaging for investigating a sample by use of the method according to claim 1, wherein the sample comprises at least a part of a body of a biological organism and contains a first marker substance and at least one further marker substance, wherein at least one of the first and the at least one further marker substance is coupled to at least one of active substance molecules and ligand molecules which are provided for a specific interaction with the sample, comprising:
an X-ray radiation source device which is arranged for irradiation of the sample with X-ray radiation, wherein X-ray fluorescence of the first marker substance and of the at least one further marker substance is excited, and fluorescence lines of the X-ray fluorescence of the first and the at least one further marker substance are different,
a detector device which is configured for spatially and spectrally resolved detection of the X-ray fluorescence of the first marker substance and the at least one further marker substance, and
an evaluation device which is configured for determination of a spatial distribution of the first marker substance and in addition a spatial distribution of the at least one further marker substance in the sample, from the detected X-ray fluorescence.

27. A marker substance kit which is configured for introducing marker substances into a sample for multi-parametric X-ray fluorescence imaging on the sample, comprising
a first marker substance which emits X-ray fluorescence upon irradiation with X-ray radiation, and
at least one further marker substance which emits X-ray fluorescence upon irradiation with X-ray radiation, wherein
fluorescence lines of the first and the at least one further marker substance are different, and
the first and the at least one further marker substance exhibit fluorescence probabilities, attenuations of the X-ray fluorescence in the sample, and background noise levels on account of scattering in the sample which are the equal or are similar to such an extent that the detection of the X-ray fluorescence at equal concentration of the marker substances results in comparable statistical significance levels, which can be maximized by use of a selection of the irradiation energy.

28. The method according to claim 19, wherein
the marker molecules are bound to at least one of active substance and ligand molecules, the marker molecules in each case comprising one of the first and at least one further X-ray fluorescence element.
